(19) United States
(12) Patent Application Publication
(10) Pub. No.: US 2023/0201501 A1
(43) Pub. Date: Jun. 29, 2023

(54) IMPROVEMENTS TO AN ASSISTED VENTILATION INTERFACE

(71) Applicant: SOUTHMED LIMITED, Dunedin (NZ)

(72) Inventors: Richard James AIMERS, Mosgiel (NZ); Andrew WALLACE, Saint Leonards (NZ); William Maynard EARLY, Brighton (NZ); Matthew Richard LEAPER, Mosgiel (NZ); Geoffrey Mark SHAW, Fendalton (NZ); Alastair Craig CARR, Dunedin (NZ)

(21) Appl. No.: 17/979,376

(22) Filed: Nov. 2, 2022

Related U.S. Application Data
(63) Continuation of application No. PCT/NZ2021/050080, filed on May 7, 2021.

(30) Foreign Application Priority Data
May 7, 2020 (NZ) 764290
Jul. 13, 2020 (NZ) 766142

(51) Int. Cl. A61M 16/00

(52) U.S. Cl. CPC A61M 16/009; A61M 16/0003; A61M 16/0627

(57) ABSTRACT

Described herein are improved assisted ventilation interfaces along with methods and uses thereof. The interfaces comprises a hood embodiment with altered design aspects to decrease or even avoid the risk of leakage and potential viral transmittance along with providing other benefits. In one aspect, the patient interface comprises a hood with a free-breathing valve and an integral viral filter. The free-breathing valve and viral filter may be separate to or integral to the exhaust port. In a further embodiment multiple branches may be used from the exhaust port with multiple viral filters. In a further embodiment, an internal pressure gauge may be used. In a further embodiment dual air sources may be used.

### RELATED APPLICATIONS

This application is a continuation if International Patent Application No. PCT/NZ2021/050080, filed May 7, 2021, which claims benefit of priority from New Zealand Patent Application No. 766142, filed Jul. 13, 2020, and New Zealand Patent Application No. 764290, filed May 7, 2020, the contents of which are incorporated herein by reference.

### TECHNICAL FIELD

Described herein are improvements to an assisted ventilation interface. More specifically, a hood is described with an integrated free-breathing valve and viral filter; a hood with an internal pressure gauge; and a hood with dual air sources. Associated methods and uses are also described.

### BACKGROUND ART

Assisted ventilation interfaces are widely used to deliver air/oxygen/gases/fluids to a patient (human or non-human animals) in need thereof.

The ventilation interfaces (termed a ‘patient interface’ hereafter) directs air, oxygen or other breathing gas/fluid from a source (e.g. a ventilator, CPAP device or NIV device) to the patient.

Art patient interfaces may be classed as being invasive or non-invasive.

Invasive interfaces use tubes to deliver air to the patient's lungs and are commonly used when the patient is unconscious or sedated. They are inherently more involved interfaces requiring trained medical staff to apply, use and remove.

Non-invasive interfaces are typically used when the patient is conscious. Existing interfaces usually are either a mask that the patient wears over their nose and mouth, a hood that encloses the patients head from the neck upwards, or nasal cannula that delivers air only to the patient's nose.

Masks are problematic in that they can be uncomfortable if worn over extended time periods as for example they may be uncomfortable and even cause pressure sores to the patient. Masks also require careful fitting to ensure a good seal and it is easy to break a seal between the mask and patient's face which can lead to loss of pressure and viral transmittance to the external environment from the patient.

FIG. 1 shows a typical prior art respiratory hood 1. The respiratory hood comprises a hood 2 which is attached to a flexible neck seal 3 that forms a seal around the patient's neck. The hood 2 may be transparent or may have at least a transparent portion. An inlet port 4 is typically connected to a ventilator or other source of respiratory air such as a CPAP machine. An outlet port 5 allows spent air to exit from the hood 1 to ambient through a pressure regulator (not shown).

Sone respiratory hoods also contain a free-breathing valve 6. This valve is an important feature which will open a breathing pathway to outside of the hood 1 in the event that air pressure within the hood 1 drops below a set point to allow the patient to continue breathing normally. This may occur for example if the ventilator is turned off or fails. The air pressure set point may be in the range of 1 to 3 cm H2O.

Typically a free-breathing valve consists of a feature such as a disk, membrane or cone in proximity to an opening in the outside of the hood 2, which may be floating on a pin, or hinged from the side of the opening, and which in normal operation of the ventilator is held closed against the opening by the gas pressure within the hood 1. The valve 6 may also include a spring of known strength which acts against the closure of the valve, but is too weak to open it under normal ventilator pressure. When the pressure within the hood drops below the set point, the valve is then able to open under the force of the spring allowing the patient to breathe normally.

Hoods provide more comfort to the patient since there is no direct pressure on their face or other sensitive areas and the seal usually provided as a collar about the patients neck, may be made from materials compliant with the skin. This seal may provide some degree of movement to the patient allowing them to move at least somewhat without the hood seal breaking and the better seal retains a higher interface internal pressure than is possible using a face mask. Art hoods however, have inherent design risks associated with viral transmittance from the hood in the event of a loss in pressure where the hood has a free-breathing valve or when changing viral filters. Art hoods also comprise inherent risk of pressure loss or viral transmittance from existing pressure gauging/sensing. Further, because the hood has a large free air volume (dead space or volume), much of the air in the hood will be exhausted from the hood without being inhaled by the patient. This means that a significantly larger volume of air is needed for a hood relative to a mask for example which has a much smaller free air volume. High rates of air use in a hood may not be desirable for example where an oxygenated supply is required by the patient since much of the oxygenated supply is not inhaled by the patient and in effect wasted. Air flow through a hood is not as directed or targeted as via a face mask or cannula.

Art hoods also can only supply one pressure of air to the patient (the hood pressure). They are unable to provides separate air supplies or separate pressure rates of air to the patient.

Nasal cannula incorporate two nasal probes to deliver air/oxygen directly to the patient's nose/nostrils. The air flow through cannula is not intended to fully supply the patient but instead supplement natural breathing by the patient and is a useful means of increasing the level of oxygen a patient receives if the nasal cannula supply oxygenated air to the patient. Smaller air flows and pressures are required since the air flow is moved directed to the patient's nose/nostrils which minimises air required. Nasal cannula are however entirely open to the environment and have no capacity to contain viral transmittance from the patient. Further, nasal cannula have no capacity to effectively seal on the patient (e.g. open mouth), hence cannot achieve a positive pressure to assist in inflating the lungs.

It may an advantage to address at least some of the above problems with existing assisted ventilation interfaces or at least provide the public with a choice.

Further aspects and advantages of the assisted ventilation interfaces, methods and uses thereof will become apparent from the ensuing description that is given by way of example only.

### SUMMARY

Described herein are improved assisted ventilation interfaces along with methods and uses thereof. The interfaces comprises a hood embodiment with altered design aspects to decrease or even avoid the risk of leakage and potential viral transmittance along with providing other benefits that will be described further below.

In a first aspect, there is provided a patient interface comprising:
a hood comprising an interior, the hood configured to fit over the patient's head so that the patient's head or at least their nose and mouth are located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
at least one free-breathing valve separate to the exhaust port that is configured to automatically open to the environment or enclosure external to the hood in the event that the hood pressure falls below a pre-determined level; and
integral to the at least one free breathing valve is at least one viral filter located in series with the at least one free-breathing valve and before the environment or enclosure external to the hood.

In a second aspect there is provided a patient interface comprising:
a hood comprising an interior, the hood configured to fit over the patient's head so that the patient's head or at least their nose and mouth are located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
at least one free-breathing valve integral to the exhaust port that is configured to automatically open to the environment or enclosure external to the hood in the event that the hood pressure falls below a pre-determined level; and
at least one viral filter located in series with the at least one free-breathing valve and before the environment or enclosure external to the hood.

In a third aspect there is provided a patient interface comprising:
a hood comprising an interior, the hood configured to fit over the patient's head so that the patient's head or at least their nose and mouth are located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
a pressure sensor, wherein the pressure sensor is fully contained within the hood interior and wherein the pressure sensor measures absolute pressure inside the hood.

In a fourth aspect, there is provided a patient interface comprising:
a hood comprising an interior, the hood configured to fit over the patient's head so that the patient's head or at least their nose and mouth are located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
a second inlet port configured to communicate air from a second air source to, the second inlet port being fluidly connected to a nasal delivery means located in the hood interior, the nasal delivery means configured to interface with a patient's nostrils and deliver the second air source to the patient's nose.

In a fifth aspect, there is provided a method of treatment of a patient requiring breathing assistance comprising:
providing a patient interface substantially as described above;
fitting the patient interface to the patient;
providing an inlet air flow to the hood via the air inlet.

In a sixth aspect, there is provided the use of a patient interface substantially as described above in providing to a patient in need thereof, breathing assistance.

One advantage of the above patient interfaces, methods and uses thereof lies in prevention or avoidance of leakage from the interface and minimisation of the potential for viral transmittance. Other advantages also exist that are described in more detail below.

### BRIEF DESCRIPTION OF THE DRAWINGS

Further aspects of the assisted ventilation interfaces, methods and uses thereof will become apparent from the following description that is given by way of example only and with reference to the accompanying drawings in which:

FIG. 1 illustrates a perspective view of a prior art hood patient interface;

FIG. 2 illustrates a side view of one embodiment of the patient interface in the form of a hood with an exhaust port comprising a free-breathing valve and viral filter;

FIG. 3 illustrates a front view of the above patient interface with a modified exhaust post comprising two branches and two viral filters;

FIG. 4 illustrates a front view of a patient interface illustrating the use of an internal pressure gauge within the hood; and

FIG. 5 illustrates a perspective view from the hood front of a hood comprising a dual air supply.

### DETAILED DESCRIPTION

As noted above, described herein are improved assisted ventilation interfaces along with methods and uses thereof. The interfaces comprises a hood embodiment with altered design aspects to decrease or even avoid the risk of leakage and potential viral transmittance along with providing other benefits that will be described further below.

For the purposes of this specification, the term ‘about’ or ‘approximately’ and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1% to a reference quantity, level, degree, value, number, frequency, percentage, dimension, size, amount, weight or length.

The term ‘substantially’ or grammatical variations thereof refers to at least about 50%, for example 75%, 85%, 95% or 98%.

The term ‘comprise’ and grammatical variations thereof shall have an inclusive meaning—i.e. that it will be taken to mean an inclusion of not only the listed components it directly references, but also other non-specified components or elements.

The term ‘air’ or grammatical variations thereof is referred to for ease of reference however other gases/fluids may be supplied to the patient via the patient interface

The term ‘exhaust air’ or grammatical variations thereof is referred to for ease of reference. It should be appreciated that the exact composition of the exhaust air from the hood may vary from the inlet air or other inlet gases/fluids at least in part due to natural breathing gas variation from a patient. The exhaust air may for example comprises great levels of carbon dioxide or moisture than ‘air’ in a pure or ambient form.

Hood with Free-Breathing Valve and Integrated Viral Filter

In a first aspect, there is provided a patient interface comprising:
a hood comprising an interior, the hood configured to fit over the patient's head so that the patient's head or at least their nose and mouth are located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
at least one free-breathing valve separate to the exhaust port that is configured to automatically open to the environment or enclosure external to the hood in the event that the hood pressure falls below a pre-determined level; and
integral to the at least one free breathing valve is at least one viral filter located in series with the at least one free-breathing valve and before the environment or enclosure external to the hood.

The term ‘integral’ and grammatical variations thereof in the context of the above embodiment refers to the free-breathing valve comprising at least one viral filter in the free-breathing air flow path that air passing through the free-breathing valve must pass through before passage to an environment or enclosure external to the hood.

Hood with Integral Free-Breathing Valve and Viral Filter

In a second aspect, there is provided a patient interface comprising:
a hood comprising an interior, the hood configured to fit over the patient's head so that the patient's head or at least their nose and mouth are located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
at least one free-breathing valve integral to the exhaust port that is configured to automatically open to the environment or enclosure external to the hood in the event that the hood pressure falls below a pre-determined level; and
at least one viral filter located in series with the at least one free-breathing valve and before the environment or enclosure external to the hood.

The term ‘integral’ and grammatical variations thereof in the context of the above embodiment refers to the refers to the exhaust port comprising at least one free-breathing valve located on or about the exhaust port flow path that exhaust air must pass through.

As noted above, a hood is described which has a free-breathing valve and viral filter integrated together, the free-breathing valve located either separate to or with the exhaust port from the hood.

As noted too, a viral filter is also integral to either the free-breathing valve or the exhaust meaning that if the pressure drops and the free-breathing valve operates, exhaust air is filtered via the viral filter. In art hoods that have free-breathing valves, if the pressure drops and the free-breathing valve actuates, patient air breathed out passes through the free-breathing valve directly to an external environment e.g. the room in which the patient is located, and as a result viral transmittance can occur to others in the room such as medical staff.

Hood

The hood may comprise a flexible seal about an opening that fits against the patient body and seals the hood interior from the external environment. The hood and associated parts during normal operation form a closed system of airflow.

The hood may be substantially transparent, at least about the patient's face. In selected embodiments, the hood sides may be made from a transparent material such as a clear plastic. The top or bottom of the hood may be manufactured from a non-transparent material although this is not essential.

The exhaust port and integral free-breathing valve may in one embodiment be located about the front of the hood when worn by the patient.

Air Source

The air source communicated to the patient interface air inlet may be sourced from a ventilation apparatus such as a ventilator, CPAP device or NIV device.

The air itself that is communicated to the air inlet may be varied depending on patient requirements and based on the source of air available. For example, the air communicated may be ambient air, filtered air, oxygen, oxygen enriched air, air or oxygen mixed with other gases, air or oxygen mixed with other fluids e.g. steam.

Hood Pressure

When air is communicated to the hood interior, the hood interior may have a positive or higher than an external environment outside the hood. The pressure at the air inlet may be higher than the pressure at the exhaust port. The exact pressure inside the hood may be variable during normal operation. The actual pressure inside the hood may be dependent for example on the rate of air communicated to the hood from the air source. In one embodiment, the hood pressure during normal operation may be 1, or 2, or 3, or 4, or 5, or 6, or 7, or 8, or 9, or 10, or 11, or 12, or 13, or 14, or 15, or 16, or 17, or 18, or 19, or −20 cm H₂O. The hood pressure may range from 1-20 cm H₂O or from 4 to 20 cm H₂O or from at least 3 cm H₂O. It should be appreciated however that higher or lower pressures may be used.

External Environment

The environment or enclosure external to the hood noted above may be an unpressurised atmosphere

The unpressurised atmosphere may for example be the ambient pressure or room pressure in which the patient is located. Alternatively, an environment or enclosure external to the hood that exhaust air is directed to has a lower pressure than the hood interior pressure.

A key reason for positive pressure in the hood interior relative to the environment or enclosure external to the hood may be to inflate the lungs allowing a greater number of alveoli to be effective at transferring oxygen to the bloodstream. This then may lead to a reduction in patient death rates via assisted breathing and doing so in a more effective means.

Another reason for having a positive pressure in the hood interior during operation may be to urge exhaust air and any particles therein from the hood and to avoid the hood collapsing or tending to a vacuum pressure.

Free-Breathing Valve Actuation

The free-breathing valve may automatically open below the pre-determined pressure level and automatically close above the pre-determined pressure level. In one embodiment, the pre-determined pressure level in the hood at which point the free-breathing valve closes automatically is approximately 1, or 2, or 3 cm H₂O or in the range from 1-3 cm H₂O.

Free-Breathing Valve Location

As noted above, the free-breathing valve may be separate to an exhaust port with an integral viral filter or may be integral to the exhaust port again also with a viral filter. In either case, the free-breathing valve provides a free flow path to an external environment when the free-breathing valve is actuated to open (automatically or manually). In the event that the free-breathing vale is integral to the exhaust port, a bypass may be included to a PEEP valve to pressure regulator valve. This valve may be fitted by a clinician.

Free-Breathing Valve Manual Bypass

In one embodiment, the free-breathing valve may incorporate a manually actuated bypass. The manually actuated bypass may be used to manually actuate free-breathing valve opening and closure. In one embodiment, manual actuation causes full opening and/or full closure of the free-breathing valve. That is, manual actuation does not lead to a free-breathing valve part open orientation. Manual actuation may be used when the hood is fitted or removed from the patient and prior to a positive internal hood pressure developing. Manual actuation to an open position may also be useful in the event that the free-breathing valve fails or does not operate as intended in the event of a pressure drop below a pre-determined range. Manual closure may also be important to allow pressure build up in the hood once an air source is communicated to the hood via the air inlet.

Multiple Viral Filters/Tap/Branches

The exhaust outlet may comprise two or more viral filters. In one embodiment, each viral filter used may be located on a separate branch extending from the exhaust port and each branch extending from a point after the exhaust port and after a free-breathing valve if a free-breathing valve is present in the exhaust air flow i.e. exhaust air flows through the exhaust port, optionally into the free-breathing valve if present, and subsequently through a branch or branches. Branches may stem from a junction such as a Y-shape or T-shape tube that may split the flow of exhaust air.

Flow through a particular viral filter or viral filters may be governed by a tap, the tap being configured to direct exhaust air to at least one selected branch. In one embodiment, the tap selectively opens flow of exhaust air to one branch and one viral filter whilst simultaneously or near simultaneously closing fluid communication to the (or other) alternate branches and viral filters. The tap described may be configured to be biased position where one branch is fully open to exhaust air flow while alternative branches may be fully closed or blocked preventing exhaust air flow from entering the closed branches.

In one embodiment, the tap may take the form of a button, switch or toggle. The button, switch or toggle may be located on or about where a branch extends from the exhaust port or the tap may be located immediately prior to where a branch extends from the exhaust port.

Viral Filter Housing

The viral filter may be located in a housing and the housing may be fitted to, or immediately after, or after, the free-breathing valve either about the exhaust port or the separate free-breathing valve described in the above embodiments. In an alternative embodiment, the viral filter may be located in a housing and the housing formed integrally with the free-breathing valve again, either about the exhaust port or the separate free-breathing valve described in the above embodiments.

Viral Filter Location

As noted above, the viral filter may be located in series with the at least one free-breathing valve and before the environment or enclosure external to the hood. In one embodiment, the viral filter is located after the at least one free-breathing valve. In an alternative embodiment, the viral filter is located before the free-breathing valve.

In an embodiment where the free-breathing valve and viral filter are integral to the exhaust port and an exhaust air flow, the viral filter may be located at a point in the exhaust air flow path before the exhaust air flow reduces significantly in pressure below that measured at the exhaust port. The viral filter may be located in the exhaust air flow at a point before the exhaust air flow reaches the pressure of the environment or enclosure external to the hood.

Hood with an Internal Pressure Gauge

In a third aspect, there is provided a patient interface comprising:
a hood comprising an interior, the hood configured to fit over the patient's head so that the patient's head or at least their nose and mouth are located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
a pressure sensor, wherein the pressure sensor is fully contained within the hood interior and wherein the pressure sensor measures absolute pressure inside the hood.

As noted above, the patient interface described may comprise a pressure sensor that is entirely contained within the hood and which measures the absolute pressure inside the hood.

Pressure Sensor

Some art respiratory hoods may include a pressure sensing gauge to display the pressure within the hood and hence verify correct ventilation system operation. These pressure sensing gauges are typically fitted to the exhaust port of the hood, which provides a reasonable representation of the pressure within the hood. However, many pressure gauges used in medical applications are of a type which tee into the side of the exhaust line and have an opening to the ambient environment on the other side of the indication unit which gives a comparative reading to ambient pressure (measures relative pressure). This opening can allow exhaust air to escape which can in turn transmit viral matter. This is not ideal and hence having a pressure sensor wholly inside the hood is far preferable as this entirely avoids risk of air loss and viral transmittance.

An alternative art embodiment is to fit the pressure gauge after a viral filter, ensuring that only filtered air passes through the pressure gauge. This is not ideal as there is a significant pressure drop across a viral filter and this pressure drop only increases as the viral filter becomes soiled. The measured pressure therefore changes over time. In addition, a pressure gauge located after a viral filter is also less sensitive in this position to variations in pressure within the hood since the pressure gauge is sheltered from changes by the viral filter pressure drop. This form of pressure measurement is therefore not as effective in indicating how well the patient is breathing. Again, the use of a pressure sensor that is located inside the hood avoids these issues and provides a very sensitive method of detecting even small changes in patient breathing.

The pressure sensor may not be in fluid communication with the environment or enclosure external to the hood.

The pressure sensor may comprise a visual, auditory or vibratory signal indicating sensed pressure in the hood interior.

The pressure sensor output may be viewed through the hood wall.

The pressure sensor may send a signal to a device external of the patient interface that indicates the measured pressure in the hood interior.

Hood with Dual Air Sources

In a fourth aspect, there is provided a patient interface comprising:
a hood comprising an interior, the hood configured to fit over the patient's head so that the patient's head or at least their nose and mouth are located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
a second inlet port configured to communicate air from a second air source to, the second inlet port being fluidly connected to a nasal delivery means located in the hood interior, the nasal delivery means configured to interface with a patient's nostrils and deliver the second air source to the patient's nose.

Nasal Delivery Means

The nasal delivery means may a device or part(s) that enable fluid communication to the nasal passages or nostrils of the patient. In one embodiment, the nasal delivery means may nasal cannula. In a further embodiment, the nasal delivery means may be a nasal mask. Reference is made hereafter interchangeably to the nasal delivery means being nasal cannula however this should not be seen as limiting as the same function may be achieved using other nasal delivery means.

As noted above, a potential disadvantage of a respiratory hood alone is that a large amount of oxygenated air must be administered as a large proportion of the air flowing into the hood is exhausted again without being inhaled. This is a particular problem when the oxygen supply is limited such as in vehicular transportation of patients and in epidemic situations.

A nasal cannula in this case may refer to a typical device comprising two nasal probes to deliver oxygenated air directly into the patient's nostrils or nasal passages to supplement the air they are naturally breathing through their nose and mouth. Note again that a nasal mask or other nasal delivery means could also be used in a similar manner.

In the context of the patient interface described above, the combination of a hood and nasal delivery means overcomes the issue of high oxygen use in a respiratory hood since the oxygenated air supply may be directed via the cannula/nasal delivery means to the patients nostrils and, due to the hood, any viral transmittance from an infected patient is avoided. This patient interface therefore may have the advantages of both a hood and cannula/nasal delivery means whilst avoiding the common disadvantages associated with each interface alone.

In one embodiment, the nasal delivery means may be permanently attached to the second inlet port. In an alternative embodiment, the nasal delivery means may be removably attached to the second inlet port.

The patient interface may be configured so that the second inlet port and nasal cannula/nasal delivery means receive a different flow rate of air than the first inlet port and hood interior receives. That is, the amount, volume or rate of air flow communicated to the hood may be at a lower (or optionally higher) amount, volume or rate through the cannula/nasal delivery means supply than through the hood supply. The second inlet port or nasal delivery means entry port may, for example, receive a lower flow rate of air equivalent to a pressure or less than 1 cm H₂O. The first inlet port or hood entry may for example receive a higher flow rate of air equivalent to 1-3 cm H₂O pressure.

In one embodiment, the second inlet port may receive oxygenated air or an air supply different to an ambient air composition.

The first inlet port may receive ambient air.

Method of Treatment

In a fifth aspect, there is provided a method of treatment of a patient requiring breathing assistance comprising:
providing a patient interface substantially as described above;
fitting the patient interface to the patient;
providing an inlet air flow to the hood via the air inlet.

Use Treatment

In a sixth aspect, there is provided the use of a patient interface substantially as described above in providing to a patient in need thereof, breathing assistance.

The patient in the above methods and uses may be a human. Non-human animals are not excluded however and the patient interface may be useful for veterinary applications as well.

Advantages

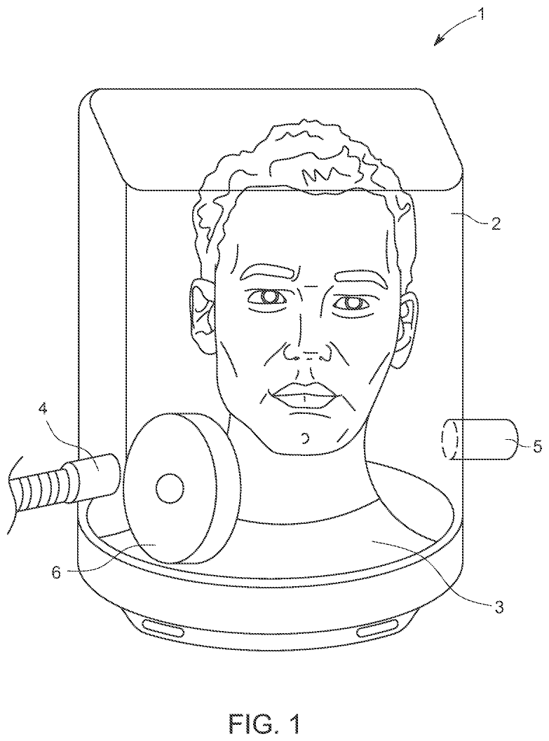

As may be appreciated from the above description, the patient interfaces described provide a number of advantages. More specifically, advantages of the above include but may not be limited to, one or more of the following:
To provide a patient interface that has the advantages of art hoods such as comfort, easy fitting, and has the reliability in terms of air/oxygen transfer to a patient like art hoods but which also solves hood problems associated with air leaking and viral transmittance.
To provide a patient interface design that reduces the number of parts required to manufacture and operate the interface. By way of example, the art hood shown in FIG. 1 requires a separate free-breathing valve whereas the improved patient interfaces described herein integrates the free-breathing valve with the exhaust port and therefore lower the part count and penetrations needed in the hood.
Optionally, the patient interfaces described removes the need for a separate externally vented free-breathing valve or the free-breathing valve has a viral filter integrated therein. This reduces risk of viral transmittance since art free-breathing valves vent to the external environment.
In an embodiment where the free-breathing valve is included as part of the exhaust port, by removing the separate free-breathing valve, it is easier to manufacture the hood and provide the free-breathing valve as a separate part to the exhaust port hence making it easier to change out parts when needed.
The patient interfaces described provide for integrated viral filtering with the free-breathing valve. This avoids the art scenario of venting via the valve to the external environment and potential for viral transmittance. Viral filtration also ensures that any transfer of viral matter via the exhaust is caught or minimised.
Where an integrated pressure valve is used, the pressure measured may be more accurate than art systems since the pressure sensor is directly measuring the absolute pressure inside the hood and not indirectly by comparison to an external environment.
An internal pressure sensor also avoids a further risk in art devices from possible viral transmittance. Art pressure sensors may be external to or vent externally from the hood and as a result, may be a source of viral transmittance. With an internal pressure sensor, this risk is eliminated.
The patient interfaces described may allow the option of changing viral filters without needing to stop patient airflow and prevent transmittance of exhaust air from the hood during viral filter changing. By way of example, where a branched exhaust is used, one branch may be open while other branches are closed. The viral filter(s) in the closed branch(es) may be removed and replaced without interfering with normal airflow through the open branch.
In embodiments where a more directed air flow is desired or where a dual air flow is desired e.g. to deliver oxygenated air and normal air to a patient, the patient interface described herein may be modified to allow for cannula delivery of air/oxygen to the patient's nostrils. Cannula delivery is known however, by using the patient interface described, delivery via a cannula can be achieved without risk of viral transmittance which is not possible from just a cannula alone.
Further to the above, a directed air flow e.g. of oxygenated air via a cannula to the patient, may reduce oxygen (or other gas/fluid) wastage by delivering the directed air flow more directly to where it is required.

The embodiments described above may also be said broadly to consist in the parts, elements and features referred to or indicated in the specification of the application, individually or collectively, and any or all combinations of any two or more said parts, elements or features.

Further, where specific integers are mentioned herein which have known equivalents in the art to which the embodiments relate, such known equivalents are deemed to be incorporated herein as if individually set forth.

### WORKING EXAMPLES

The above described improved assisted ventilation interfaces along with methods and uses thereof are now described by reference to specific examples.

### Example 1

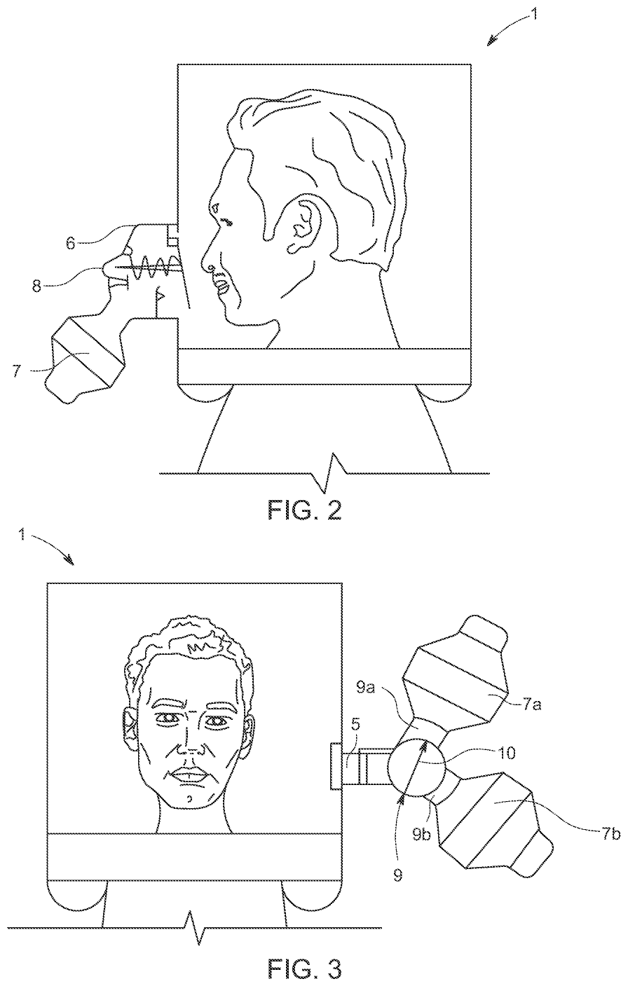

FIG. 2 shows a side view of an embodiment of a respiratory hood 1 with an embodiment of free-breathing valve 6 on the front. An interface 8 (e.g. a button, switch, or toggle) which allows the valve to be manually opened or closed irrespective of the pressure within the respiratory hood 1 may be incorporated into the free-breathing valve 6 to allow clinical staff or the patient to manually control the position of the valve. For example, the free-breathing valve 6 may be manually opened if the pressure within the respiratory hood 1 is uncomfortably high. The interface 8 may also be used to manually close the free-breathing valve 6 to allow pressure to build up within the respiratory hood 1 when the ventilator is first started.

An embodiment of the free-breathing valve 6 shown in FIG. 2 includes a viral filter 7. The viral filter 7 is positioned on an outlet of the free-breathing valve 6 leading to atmosphere. The fitment or seal between the outlet of the free-breathing valve 6 and the viral filter 7 is at least substantially air-tight so that pressured air passing through the free-breathing valve 6 from within the respiratory hood 1 to the ambient atmosphere must first pass through the viral filter 7. In this way, if the free-breathing valve 6 opens to the treatment room in the way described above, any viruses within the expelled air will be at least substantially removed by the viral filter 7 and will not escape the hood 1. This reduces the risk of high pressure virally contaminated gases escaping from the respiratory hood 1 into the room.

The viral filter 7 may be within a housing which is integrally formed with the body of the free-breathing valve 6. In other embodiments, the viral filter 7 may be modular and may fit or seal against the body or outlet of the free-breathing valve 6. These embodiments have the advantage of allowing a viral filter to be retrofitted onto an existing free-breathing valve. The viral filter 7 is replaceable so that a soiled filter can be swapped out for a fresh filter.

The viral filter 7 may be a standard viral filter used in other medical applications, such as a HEPA (high-efficiency particulate air) filter. The viral filter 7 may also meet international standards for medical use, such as ISO 5356-1 (relating to equipment used with respiratory devices.) The viral filter 7 may have a filtering efficiency of at least 95%, although preferably has a greater filtering efficiency such as at least 98%. The viral filter 7 may be capable of filtering particles of a range of sizes. As a non-limiting example, the viral filter 7 may be capable of filtering out particulates sized between 0.01 to 500 μm. Furthermore, although the term ‘viral’ filter is used in this specification, the viral filter 7 may be capable of filtering out bacteria or other pathogens. The characteristics of the viral filter 7 used in a given application may vary, and may depend at least partially on the patient's needs and condition, and the judgement of the attending clinician. These viral filter details are provided by way of example however, the patient interfaces described herein may be used with any existing viral filter and hence, these characteristics may be varied to suit the desired application of the patient interface.

FIG. 3 shows an embodiment of an exhaust port 5 of a respiratory hood 1. In the shown configuration, the exhaust port 5 leads to (i.e. is in fluid communication with) a junction 9 which directs the exhausted gas into one of two separate branches, 9a and 9b. The junction 9 contains a tap 10 which selectively blocks one of the branches 9a or 9b from the exhaust port 5 while exposing the other branch 9a or 9b to the exhaust port 5. In other words, the tap 10 is configured to selectively allow fluid communication between one of the branches 9a or 9b and the exhaust port 5. This allows clinical staff to select which of the two outlets is in use at any one time by using tap 10.

Each branch 9a and 9b includes an associated viral filter 7a and 7b. The housing of these viral filters 7a and 7b may be modular and may be fitted to each branch 9a or 9b, or may be integrally formed with branches 9a and 9b.

Viral filters must be replaced on a regular basis to ensure their effectiveness as these may become clogged. A typical replacement frequency is every 24 hours, which may be a shorter period of time than the patient is required to remain on ventilation. In a traditional respiratory hood, the removal of a viral filter allow the escapement of virally contaminated gases. The standard way to manage this is to turn off the ventilator before the filter is removed in order to reduce the pressure within the hood. However this may not completely eliminate pressure and contaminated gases may still escape. Additionally, the removal of ventilation may be detrimental to the patient.

In the configuration shown in FIG. 3 either of viral filters 7a or 7b may be replaced without stopping the ventilator and without exposing the room to unfiltered gases. As an illustrative example, if the viral filter 7a is to be replaced and is currently in use, the tap 10 is turned to close off the branch 9a. The ventilator continues to operate as normal with gas being exhausted through branch 9b and associated viral filter 7b. The viral filter 7a is then safely removed with the tap 10 still directing exhaust through branch 9b. In this way, ventilation to the patient is uninterrupted with no interruption to the filtration of exhausted gases. Viral filter 7b can then also be replaced if necessary by repeating the process (switching the tap 10 to block branch 9b and exhausting gas through the newly-replaced viral filter 7a.)

In some embodiments, any residual high pressure gases within the branch downstream of the tap 10 may be bled through its associated viral filter before the viral filter is removed. In other embodiments, the viral filter may be positioned substantially immediately after the tap, and a bleeding step may not be necessary.

The tap 10 may be biased by a spring or other mechanism (not shown) so that it is only stable in positions corresponding to the closure of either (or any) of the branches. Referring to the example embodiment depicted in FIG. 3, the tap 10 may be configured so that it is always urged into a position where one branch 9a or 9b is completely closed, while the other branch is completely open. In these embodiments, if the operator accidentally placed the tap in a position where each branch 9a and 9b would be partially open, the tap will snap to a position where one branch is fully closed under the action of the biasing spring or equivalent element. This may reduce the possibility that one of the branches 9a or 9b is accidentally left partially exposed to the atmosphere while a viral filter 7a or 7b is being replaced.

Furthermore, the use of 2 branches and 2 viral filters is only an illustration of the applicant's overall invention, and other embodiments could include any plurality of branches and associated viral filters. The number of branches and viral filters will depend on the application of the ventilator.

### Example 2

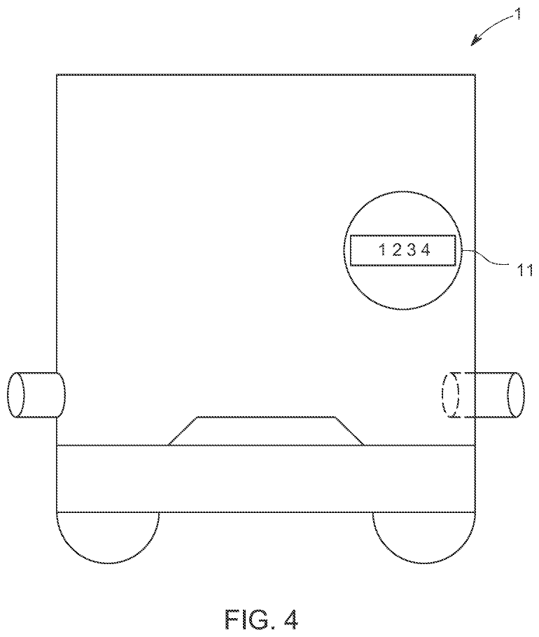

FIG. 4 shows an embodiment of a pressure sensing gauge 11. A pressure sensing gauge may be used to ensure that the ventilator is operating correctly (i.e. it has been set to the correct pressure for the particular patient). It may also indicate if there is a blockage or leak within the system for example by showing a change in pressure. When placed sufficiently close to the patient and when of a fast operating design it may also record variations in pressure that occur due to the patient's breathing, and may therefore give an important clinical perspective on the patient's wellbeing.

A particular problem with a pressure sensing gauge on this type of equipment is that it often relies on air leakage flowing past the gauge and out to atmosphere. In the case of an infectious patient this may result in viral contamination. In the configuration shown in FIG. 4, a pressure sensing unit 11 is mounted within the respiratory hood 1 which measures absolute pressure and is therefore not connected to the outside atmosphere. In other words, the pressure sensor is not in fluid communication with the atmosphere outside of the respirator hood when the hood is in use. The sensor 11 may contain a visible gauge, and it may be located within the hood 1 in a convenient location to allow clinical staff to view the gauge from outside of the hood 1. In the configuration shown, the sensor 11 is stuck to the inside of the hood 2. In a preferred configuration the sensor 11 may also broadcast a wireless signal of its reading, e.g. by Bluetooth, and this signal may be readable on an external device such as a mobile phone or tablet (that is to say, an external device may be able to interface with the sensor 11). This may enable recording of changes in pressure over a period of time, for example to record the breathing characteristics of the patient.

### Example 4

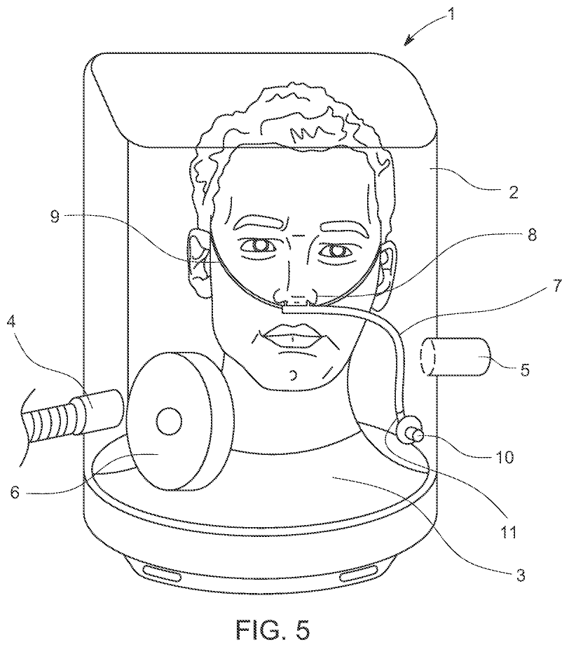

FIG. 5 shows a respiratory hood similar to the one shown in FIG. 1. In this embodiment a nasal cannula 7 may be fitted to the patient via a band 9 around the patient's head. Nasal probes 8 on the cannula 7 are inserted into the patient's nostrils. The inlet end of the cannula 7 has a connector 10. The connector 10 is fitted through port 11 in the hood 1 to form an entry point in the hood 1 for oxygenated air to the nasal cannula 7.

In operation the cannula 7 will be connected to a low volumetric flow of oxygenated air via port 11, while a high volumetric flow of less concentrated air may be administered through port 4. This high volumetric flow may for example be filtered ambient air rather than a precisely metered oxygenated source. In this way a high flow of air can be administered to fulfil the operating characteristics of the respiratory hood 1 while conserving the oxygen supply.

Aspects of the assisted ventilation interfaces, methods and uses thereof have been described by way of example only and it should be appreciated that modifications and additions may be made thereto without departing from the scope of the claims herein.

What is claimed is:

1. A patient interface comprising:
a hood comprising an interior, the hood configured to fit over the patient's head so that the patient's head is located in the interior of the hood and wherein the hood is configured to provide an enclosed breathing environment;
an air inlet configured to communicate air from an air source to the hood interior;
an exhaust port configured to communicate air from the hood interior to an environment or enclosure external to the hood;
a second inlet port configured to communicate air from a second air source to, the second inlet port being fluidly connected to a nasal delivery means located in the hood interior, the nasal delivery means configured to interface with a patient's nostrils and deliver the second air source to the patient's nose;
and wherein the patient interface is configured so that the second inlet port receives a lower volume rate of air than the first inlet port and hood interior receives hence conserving the air supply required by the patient interface to meet a patient's oxygen requirements.

2. The patient interface as claimed in claim 1 wherein the nasal delivery means is a nasal cannula.

3. The patient interface as claimed in claim 1 wherein the second inlet port receives oxygenated air and the first inlet port receives ambient non-oxygenated air.

4. The patient interface as claimed in claim 1 wherein the patient interface further comprises at least one free-breathing valve that is configured to automatically open to the environment or enclosure external to the hood in the event that the hood pressure falls below a pre-determined level; and at least one viral filter located in series with the at least one free-breathing valve and before the environment or enclosure external to the hood.

5. The patient interface as claimed in claim 1 wherein the free-breathing valve is separate to the exhaust port.

6. The patient interface as claimed in claim 1 wherein the free-breathing valve is integral to the exhaust port.

7. The patient interface as claimed in claim 1 wherein the patient interface further comprises a pressure sensor, wherein the pressure sensor is fully contained within the hood interior and wherein the pressure sensor measures absolute pressure inside the hood.

8. The patient interface as claimed in claim 1 further comprising:
at least one free-breathing valve separate to the exhaust port that is configured to automatically open to the environment or enclosure external to the hood in the event that the hood pressure falls below a pre-determined level; and
integral to the at least one free breathing valve is at least one viral filter located in series with the at least one free-breathing valve and before the environment or enclosure external to the hood.

9. The patient interface as claimed in claim 1 further comprising:
at least one free-breathing valve integral to the exhaust port that is configured to automatically open to the environment or enclosure external to the hood in the event that the hood pressure falls below a pre-determined level; and
at least one viral filter located in series with the at least one free-breathing valve and before the environment or enclosure external to the hood.

10. The patient interface as claimed in claim 8 wherein the free-breathing valve is integral to two or more viral filters, each viral filter located on a separate branch and each branch extending from a point after the free-breathing valve, flow through either or a viral filter governed by a tap that is configured to direct air from the free-breathing valve to at least one selected branch and viral filter.

11. The patient interface as claimed in claim 9 wherein the exhaust outlet comprises two or more viral filters, each viral filter located on a separate branch and each branch extending from a point after the free-breathing valve, flow through either or a viral filter governed by a tap that is configured to direct exhaust air to at least one selected branch.

12. The patient interface as claimed in claim 10 wherein the tap selectively opens flow of exhaust air to one branch and one viral filter whilst simultaneously closing fluid communication to the or other alternate branches or viral filters.

13. The patient interface as claimed in claim 13 wherein the tap is biased to a branch fully open and branch fully closed position.

14. The patient interface as claimed in claim 1 further comprising:
a pressure sensor, wherein the pressure sensor is fully contained within the hood interior and wherein the pressure sensor measures absolute pressure inside the hood.

15. The patient interface as claimed in claim 14 wherein the pressure sensor is not in fluid communication with the environment or enclosure external to the hood.

16. The patient interface as claimed in claim 14 wherein the patient interface further comprises at least one free-breathing valve that is configured to automatically open to the environment or enclosure external to the hood in the event that the hood pressure falls below a pre-determined level; and at least one viral filter located in series after the at least one free-breathing valve and before the environment or enclosure external to the hood.

17. A method of treatment of a patient requiring breathing assistance comprising:
providing a patient interface as claimed in claim 1;
fitting the patient interface to the patient;
providing an inlet air flow to the hood via the air inlet.

18. Use of a patient interface as claimed in claim 1 in providing breathing assistance to a patient in need thereof.
